(19) United States
(12) Patent Application Publication
(10) Pub. No.: US 2006/0257883 A1
(43) Pub. Date: Nov. 16, 2006

(54) Detection and measurement of hematological parameters characterizing cellular blood components

(76) Inventors: David G. Bjoraker, Gainesville, FL (US); Richard J. Melker, Gainesville, FL (US); Donn Michael Dennis, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/125,937

(22) Filed: May 10, 2005

(51) Int. Cl. C12Q 1/68; G01N 33/92; G01N 33/00

(52) U.S. Cl. 435/6; 436/71; 436/86

(57) ABSTRACT

Systems and methods for the diagnostic analysis of blood samples. The present invention uses sensor technology useful in the analysis of headspace sample from blood to provide an efficient and accurate means for identifying the presence of a volatile marker associated with hematological diseases or conditions. In a preferred embodiment, the sensor technology of the present invention includes detecting means such as RNA oligonucleotide chains or aptamers.

### BACKGROUND OF THE INVENTION

There is a great need for the development of efficient and accurate systems for the diagnosis of a variety of medical conditions, disorders, and diseases. A means for rapid and accurate analysis of ex vivo bodily fluid samples for clinical diagnosis at the point-of-care (POC) is particularly desirable. This requires an effective means for identifying from a patient the presence of specific chemical and/or biological agents (also referred to herein as markers) such as nucleic acids, proteins, illicit drugs, toxins, pharmaceuticals, carcinogens, poisons, allergens, and infectious agents.

Warfarin is currently the most widely prescribed oral anticoagulant (OAC) in the United States and overall the 13^{th} most prescribed drug in this country. Warfarin has been used for many years to prevent and treat thromboembolic complications associated with many medical conditions such as atrial fibrillation, valvular-heart disease, and deep venous thrombosis. Although it is very efficacious, the use of warfarin is a frequent cause of severe adverse drug reactions (ADRs). Among ADRs, warfarin is the 2^{nd} most common cause of hospitalizations, primarily due to hemorrhagic complications.

The utilization of warfarin in the clinical arena is potentially complicated by a number of factors. The drug has a relatively narrow therapeutic index and requires frequent visits to outpatient clinics to monitor the level of anticoagulation. The dose requirements of warfarin can differ substantially between patients, and drug-drug interactions (DDIs) commonly modulate the level of anticoagulation to a large degree. Because warfarin is a CYP450 substrate with a long elimination half life (20 to 40 hours in man), its use has been associated with many ADRs, which can have fatal consequences. Despite these problems, the use of oral warfarin for anti-thrombotic applications has a very favorable benefit-risk ratio. It has been estimated that warfarin prevents 20 strokes for every bleeding episode that it inadvertently causes.

Warfarin causes anticoagulation by acting as an inhibitor of VKOR. VKOR is a hepatic microsomal enzyme that catalyzes the post-translational carboxylation of specific glutamyl (Glu) residues to γ-carboxyglutamates (Gla) on the N-terminal regions of vitamin K-dependent proteins. γ-Carboxylation requires the reduced form of vitamin K (vitamin KH₂). Specifically, carboxylation of vitamin K-dependent proteins requires a functional vitamin K cycle to produce the active vitamin K cofactor for the γ-carboxylase which posttranslationally modifies precursors of these proteins to contain γ-carboxyglutamic acid residues. The warfarin-sensitive enzyme vitamin K epoxide reductase (VKOR) of the cycle reduces vitamin K 2,3-epoxide to the active vitamin K hydroquinone cofactor and thus causes the liver to produce partially decarboxylated proteins with reduced coagulant activity.

The Gla-proteins critical to the blood coagulation cascade include clotting Factors II, VII, IX, and X, as well as endogenous anticoagulant proteins, termed C and S. Of the four serine protease clotting factors, Factor VII has the shortest half life in humans (6 hrs). Therefore, a reduction in Factor VII activity serves as the earliest marker to herald the onset of warfarin-induced anticoagulation in the extrinsic coagulation pathway. Subsequently, because Factor II (50 hrs) and Factor X (36 hrs) have longer half lives, reductions in the functional levels, and hence activities, of both Factor II and Factor X also decrease if VKOR inhibition persists.

Given the above information, it is not surprising that there is a significant lag time associated with warfarin dosing, effective VKOR inhibition within hepatocytes (which is dependent upon dose and hepatocyte drug levels) and clinically effective anticoagulation. The clinical anti-thrombotic efficacy of warfarin is more closely linked to reductions in Factor II and Factor X activity than to reductions in Factor VII or IX. The International Normalized Ratio (INR), which has been validated in humans, integrates these factor changes into its value and accurately measures warfarin-induced anticoagulation and predicts antithrombotic efficacy. However, due to the lag effect, the major problem with measuring INR is that it only provides an assessment of current anticoagulation status. This is problematic. For example, due to the lag time between warfarin-induced reductions in VKOR function and decreases in active vitamin K-dependent factor levels, a significant drug-drug interaction (DDI) involving the CYP450 system could acutely elevate the plasma concentration of warfarin 10× or more and cause major delayed hemorrhagic complications (in a few days), but the current INR would be normal. Likewise, for reasons cited above, the plasma levels of warfarin have also been found to correlate very poorly to the current level of anticoagulation in humans. In contrast, the accurate real time assessment of VKOR activity would be a significant addition to the clinical tools used to properly manage warfarin anticoagulation.

Current methods of detecting markers indicative of a medical condition (i.e., level of anticoagulation resulting from warfarin administration) entail extraction of a sample of bodily fluid (normally blood or urine) into organic solvents, followed by analysis using stand-alone analytical systems such as gas-liquid chromatography and/or mass spectroscopy. These methods are time-consuming and often expensive. Certain markers of interest (i.e., nucleic acids, carcinogens, or toxins) are not readily detected by standard chemical tests utilized in a typical clinical physician's office or even in hospital laboratories. Moreover, on-site test devices for accurate marker detection are not presently available. The development of a biosensor device that could accurately and efficiently detect/screen for chemical and biological agents in blood samples would therefore provide a significant cost and time benefit to society.

To date, no one has applied sensor technology to detecting and quantifying volatile markers associated with cellular components, red blood cells, platelets, and white blood cells.

### BRIEF SUMMARY

The present invention provides unique systems and methods for the detection of biomarkers associated with the components of blood in the diagnosis of medical diseases/conditions. In particular, the present invention utilizes sensor technology to detect either in exhaled breath or sample of bodily fluid headspace the presence of biomarkers or markers associated with hematological diseases/conditions.

Biomarkers detected in accordance with the subject invention include those associated with cellular components, red blood cells, platelets, and white blood cells (lymphocytes, neutrophils, eosinophils, macrophages, etc.). In particular embodiments of the invention, markers released from the cellular components of blood or from the progenitor cells of the circulating cells or from chemicals designed to react with the circulating cells of the blood can be detected in bodily fluid samples or headspace and measured for diagnostic purposes (i.e., to measure several features of blood cell populations). In certain embodiments, compositions are provided to a patient, wherein markers in the composition are made detectable in bodily fluid samples or headspace upon detection of a target biomarker.

Markers of the invention can be detected in exhaled breath, in headspace gas overlying solutions of these cells (including vapor above a sample of whole blood), and in the atmosphere overlying shed blood and bleeding wounds. Detection of such markers allows for quantification, ratio determination, maturation determination, and diagnosis of pathological alterations, such as infection (i.e., malaria, protozoal, viral, or bacterial infection) or malignancy (i.e., leukemia, lymphoma, or Hodgkin's disease).

Specific embodiments of the invention contemplate collecting a sample of blood from a patient and retaining the sample in a closed container that includes an area (also referred to herein as the headspace) for encompassing markers released from the blood sample. The headspace of markers is then exposed to a sensor of the present invention to detect and quantify at least one target marker in the headspace of the blood sample. When the sensor detects at least one target marker, it provides a detectable signal that communicates to the user not only the presence of the target marker but also, in a preferred embodiment, the quantity of the target marker in the blood sample. Notification of the presence and/or quantity of the target marker in the blood sample enables user diagnosis of the specific condition, disorder, or disease associated with the target marker.

In certain embodiments, the headspace of volatile markers is directly exposed to a sensor of the present invention within the container. In other embodiments, the headspace of volatile markers is transferred out of the container to a receptacle prior to analysis by a sensor of the present invention. In a related embodiment, either the blood sample and/or the headspace of volatile markers can be treated either prior to, during, or after transfer out of the container to concentrate the headspace. For example, the headspace of volatile markers can be treated to remove water vapor or the blood sample heated to a temperature between 50° F. to 110° F. to promote increased concentration of volatile markers in the headspace.

In other embodiments of the invention, a sample of exhaled breath is analyzed using a sensor of the invention to detect volatile markers associated with cellular components (such as red blood cells, platelets, white blood cells, etc.). According to the subject invention, detection of such markers in exhaled breath using sensor technology is fast, convenience, low in cost, and can be performed at the POC without extensive clinical training.

Further embodiments of the invention contemplate applying sensor technology to shed blood or bleeding wounds to detect volatile markers associated with cellular components for clinical diagnostic purposes.

In accordance with the present invention, the sensor includes well-known biodetectors or biosensors. Such biodetectors or biosensors include naturally occurring and/or synthetic compounds having high specificity and sensitivity to chemical and/or biological compounds of interest. Suitable biodetectors or biosensors of the invention include, but are not limited to, antibodies, proteins, and aptamers. Contemplated sensor technology includes, but is not limited to, semiconductor gas sensor technology, conductive polymer gas sensor technology, surface acoustic wave gas sensor technology, and immunoassays.

The results from sensor analysis of blood, breath, or other samples are optionally provided to the user (or patient) via a reporting means. In one embodiment, the sensor technology includes the reporting means. The reporting means can provide the results to the user (or patient) via known avenues of communication such as facsimile, electronic mail, mail or courier service, or any other means of safely and securely sending the report to the recipient.

The advantages of the invention are numerous. First, for healthcare personnel, the invention provides a method that can readily diagnose a patient's condition (or disorder/disease) based on a small sample of the patient's blood or breath. Second, the invention is inexpensive and has broad medical applications for detecting a wide range of compounds (including licit and illicit drugs) indicative of specific diseases and/or conditions of interest.

According to the present invention, hematological or blood-based diseases, disorders and/or conditions such as, but not limited to, anemias (i.e., sickle cell anemia, aplastic anemia, Diamond-Blackfan anemia, hemolytic anemia, hypoplastic anemia, refractory anemia), leukemias (i.e., erythroblastic leukemia, lymphocytic leukemia, hairy cell leukemia, monocytic leukemia, myelocytic leukemia, myeloid leukemia, nonlymphocytic leukemia), hemophilia (i.e., hemophilia A, hemophilia B), high blood cholesterol, lymphoma (i.e., large-cell lymphoma, mantle-cell lymphoma, small cleaved-cell lymphoma, T-cell lymphoma), hepatitis, HIV, anticoagulation (i.e., with the administration of warfarin), and thrombocytosis, can be detected and subsequently treated using the system and method of the subject invention.

Analysis of blood or breath samples utilizing methods disclosed herein can be applied to a wide range of POC diagnostic tests. For example, potential applications include detection of licit and illicit drugs, detection of a wide range of markers related to specific hematological diseases, and detection of any other compounds that appear in blood.

Moreover, analysis of blood samples using the systems and methods of the present invention can enable timely interventions for time-sensitive hematological conditions or diseases.

### BRIEF DESCRIPTION OF THE DRAWINGS

FIG. 1 shows a capnogram of a single respiratory cycle and a capnogram of several breaths from a patient with obstructive lung disease.

FIG. 2 is a graphical illustration of how warfarin inhibits enzyme activity to encourage anticoagulation within the body.

### DETAILED DISCLOSURE

The present invention is directed to the efficient, timely, and accurate analysis of a sample of a patient's breath or blood to detect and/or quantify markers indicative of conditions, disorders, or diseases associated with blood. The systems and methods of the invention use sensors capable of identifying and discriminating volatile markers released from the cellular components of blood, from the progenitor cells of the circulating cells, or from chemicals designed to react with the circulating cells of the blood. Commonly available sensor technology is used in accordance with the present invention to detect the presence of a volatile markers associated with blood components in the diagnosis and treatment of hematological diseases/conditions.

The cellular components of blood that can be characterized are the red blood cells (also known as erthyrocytes) including reticulocytes; blood platelets (also known as thrombocytes); white blood cells (also known as leukocytes) including lymphocytes, neutrophils, eosinophils, basophils, and monocytes; and pathological circulating blood cells including Reed-Stemberg cells, plasma cells, macrophages, and circulating malignant cells from solid tumors.

Features of the cells detected in accordance with the subject invention include, but are not limited to, their presence; their type; their concentration (such as the number of cells that are circulating per unit volume of blood); their ratio to other blood cells; their rate of formation; their maturation; their rate of removal from the circulating blood; their activity status (i.e., activated platelets); their transformation into malignant cells (i.e., lymphoblasts compared with lymphocytes, myeloblasts compared with mature polymophonuclear neutrophils); or their infection or infestation by pathogens such as a parasites (e.g., malaria), viruses, or bacteria.

In operation, a fluid sample (such as blood or exhaled breath) is collected from a patient. Blood samples are preferably collected in containers that include a means for sealing the container and are of sufficient volume to allow for the upper portion of the container to be left empty for headspace. After collecting and sealing the blood sample into the container, the blood sample is allowed to equilibrate. Sensors capable of detecting volatile markers associated with blood components in the diagnosis and treatment of hematological diseases/conditions are then applied to the headspace. Detection of one or more volatile markers in the headspace by the sensors of the subject invention is communicated to the user.

In one embodiment, compositions are provided to a patient requiring analysis of cellular components. The compositions comprise elements that react with target cellular components of blood and release a volatile marker that is detectable in exhaled breath and/or headspace from samples of blood. In a related embodiment, the elements of the composition include nanotechnology (such as capped nanotubes enclosing volatile markers) and biosensor technology (such as aptamer-based sensors used to identify target cellular components).

In another embodiment of the invention, samples of exhaled breath and/or blood are taken from a patient and endogenous volatile markers associated with cellular components are detected via sensor technology.

In certain embodiments, either the blood sample and/or the headspace of volatile markers can be treated prior to, during, or after transfer out of the container to concentrate the sample of markers in the headspace. For example, the headspace of volatile markers can be treated to remove water vapor or the blood sample can be heated to a temperature between 50° F. to 110° F. to promote increased concentration of volatile markers in the headspace.

With other embodiments of the invention, a sample of patient's exhaled breath is analyzed using sensor technology to detect the presence of volatile markers associated with cellular components. Detection of such markers can allow the quantification, ratio determination, maturation determination, and diagnosis of pathological alterations. In related embodiments, samples of exhaled breath can be treated to concentrate the sample of markers.

Definitions

Unless otherwise stated, the following terms used in the specification and claims have the meanings given below.

The term “aptamer,” as used herein, refers to a non-naturally occurring oligonucleotide chain that has a specific action on a target marker of interest. A specific action includes, but is not limited to, binding of the target marker, catalytically changing the target marker, and reacting with the target marker in a way which modifies/alters the marker or the functional activity of the marker. The aptamers of the invention preferably specifically bind to a target marker and/or react with the target marker in a way which modifies/alters the marker or the functional activity of the marker.

Aptamers include nucleic acids that are identified from a candidate mixture of nucleic acids. In a preferred embodiment, aptamers include nucleic acid sequences that are substantially homologous to the nucleic acid ligands isolated by the SELEX method. Substantially homologous is meant a degree of primary sequence homology in excess of 70%, most preferably in excess of 80%.

The “SELEX” methodology, as used herein, involves the combination of selected nucleic acid ligands, which interact with a target marker in a desired action, for example binding to a protein, with amplification of those selected nucleic acids. Optional iterative cycling of the selection/amplification steps allows selection of one or a small number of nucleic acids, which interact most strongly with the target analyte/biomarker from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

The term “indicator aptamers,” as used herein, refers to aptamers to which molecular beacons are attached, such as those described in U.S. Pat. Nos. 6,399,302 and 5,989,823.

The term “molecular beacons,” as used herein, refers to a molecule or group of molecules (i.e., a nucleic acid molecule hybridized to an energy transfer complex or chromophore(s)) that can become detectable and can be attached to a biodetector/biosensor under preselected conditions. For example, an embodiment of the present invention includes an aptamer-bound fluorescence beacon that (a) quenches when a target marker is reversibly bound to the aptamer and (b) is detectable with a photodetector to quantify the concentration of target marker present.

As used herein, “marker” refers to naturally occurring and/or synthetic compound that is an indicator of a condition, disease state, disorder, or a normal or pathologic process that occurs in the patient. The term “marker,” as used herein, can refer to any substance, including biomarkers or chemical and/or biological agents that can be measured in an analytical procedure.

The term “biomarker” refers to a biochemical in the body that has a particular molecular trait to make it useful for diagnosing a condition, disorder, or disease and for measuring or indicating the effects or progress of a condition, disorder, or disease. For example, common biomarkers found in a person's bodily fluids (i.e., breath or blood), and the respective diagnostic conditions of the person providing such biomarkers include, but are not limited to, acetaldehyde (source: ethanol; diagnosis: intoxication), acetone (source: acetoacetate; diagnosis: ketogenic diet; diabetes), ammonia (source: deamination of amino acids; diagnosis: uremia and liver disease), CO (carbon monoxide) (source: CH₂Cl₂, elevated % COH; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source: metabolism; diagnosis: periodontal disease/ovulation), MeS (source: metabolism; diagnosis: cirrhosis), and Me₂S (source: infection; diagnosis: trench mouth).

The term “volatile,” as used herein, refers to a molecule or compound that is present in the headspace of a liquid (i.e., blood) or gas (i.e., exhaled breath). It will be recognized that the physical state of volatility can be changed by pressure, temperature, as well as by affecting surface tension of a liquid (i.e., blood) by the presence of or addition of chemicals or compounds known by the skilled artisan (i.e., salts can affect surface tension of a liquid). According to the subject invention, both markers and biomarkers can be volatile.

Additional markers that can be detected using the present invention include, but are not limited to, illicit, illegal, and/or controlled substances including drugs of abuse (i.e., amphetamines, analgesics, barbiturates, club drugs, cocaine, crack cocaine, depressants, designer drugs, ecstasy, Gamma Hydroxy Butyrate—GHB, hallucinogens, heroin/morphine, inhalants, ketamine, lysergic acid diethylamide—LSD, marijuana, methamphetamines, opiates/narcotics, phencyclidine—PCP, prescription drugs, psychedelics, Rohypnol, steroids, and stimulants); allergens (i.e., pollen, spores, dander, peanuts, eggs, and shellfish); toxins (i.e., mercury, lead, other heavy metals, and Clostridium Difficile toxin); carcinogens (i.e., acetaldehyde, beryllium compounds, chromium, dichlorodiphenyltrichloroethane (DDT), estrogens, N-methyl-N′-nitro-N-nitrosoguanidine (MNNG), and radon); and infectious agents (i.e., Bordettella bronchiseptica, citrobacter, Escherichia coli, hepatitis viruses, herpes, immunodeficiency viruses, influenza virus, Listeria, micrococcus, mycobacterium, rabies virus, rhinovirus, rubella virus, Salmonella, and yellow fever virus).

The terms “cellular components,” “blood,” or “blood components” are used interchangeably and, as used herein, refer to the mixture of cells and materials that make up blood obtained from a patient. According to the subject invention, cells and materials that make up cellular components include, but are not limited to, erythrocytes, hemoglobin, leukocytes, thrombocytes, plasma, sugars, lipids, vitamins, minerals, proteins, hormones, enzymes, and antibodies. The terms cellular components, blood, or blood components also include experimentally separated fractions of blood.

As used herein, the term “headspace” refers to the volume left at the top of a container before sealing. According to the subject invention, a container used in handling blood samples can include, without limitation, test tubes, cuvettes, Petri dishes, cups, beakers, pipettes, jars, bags, and vials. All of the containers of the invention include a means for sealing the container and providing blood headspace sample. Contemplated means for sealing a container of the invention include, but are not limited to, plugs, covers, stoppers, and fitted caps.

The term “sensor,” as used herein, refers to the use of naturally-occurring and/or synthetic compounds as highly specific and sensitive detectors of various types of markers. Naturally-occurring compounds such as antibodies, proteins, receptor ligands, and receptor proteins have been used to provide molecular recognition for a wide variety of target molecules in diagnostic assays. Alternatively, synthetic compounds such as aptamers have been manufactured that mimic naturally occurring mechanisms of DNA, RNA, and protein synthesis in cells to facilitate detection of target volatile markers. Sensors of the invention include both chemical and physical sensors such as SAW sensors, chemoresistant polymers, sensors based on nanotechnology, and the like.

A “patient,” as used herein, describes an organism, including mammals, from which bodily fluid samples are collected in accordance with the present invention. Mammalian species that benefit from the disclosed systems and methods of diagnosis include, and are not limited to, apes, chimpanzees, orangutans, humans, monkeys; and domesticated animals (e.g., pets) such as dogs, cats, mice, rats, guinea pigs, and hamsters.

Nanoparticles

Nanostructure-based assemblies offer timely, and effective detection and notification of a disease or condition of interest. Such assemblies are based on nanoparticles, which provide a mechanism for the release of detectable volatile markers upon detection of a target biomarker. In certain embodiments of the invention, compositions comprising nanoparticles are administered to a patient requiring clinical analysis of certain blood cellular components. The nanoparticles are designed to have an affinity for a target biomarker and the capacity to release a volatile marker upon nanoparticle detection of the target biomarker.

According to the present invention, nanoparticles can be produced in a wide range of sizes and shapes, and composed of a wide range of materials, or combination of materials, optimized for in-vivo administration. Contemplated shapes include, but are not limited to, spherical, elliptical, cubic, cylindrical, tetrahedron, polyhedral, irregular-prismatic, icosahedral, and cubo-octahedral forms. Nanoparticles intended for in-vivo use are of any dimension, preferably with a maximum dimension less than 500 nm, so as to ensure proper distribution at the microvasculatoure level, without any occlusion of blood flow. More preferably, the nanoparticles of the subject invention are of a maximum dimension less than 100-150 nm. The “maximum dimension” of a nanoparticles is the maximum distance between any two points in the nanoparticle. In a preferred embodiment, the nanoparticles are in the form of tubular bodies (also known as “nanotubes”), which are either hollow or solid and include either open ends or one or both closed ends.

In accordance with the present invention, the nanoparticle-based assemblies are composed of biodegradable substances. In other embodiments, the nanoparticle-based assemblies of the invention are composed of biocompatible substances.

Methods of preparation of nanoparticles are well known in the art. For example, the preparation of monodisperse sol-gel silica nanospheres using the well-known Stober process is described in Vacassy, R. et al., “Synthesis of Microporous Silica Spheres,” J. Colloids and Interface Science, 227, 302 (2000).

Nanoparticles, in accordance with the present invention, can be prepared from a single material or a combination of materials. For example, nanotubes can be prepared from either one or a combination of materials including, but not limited to, polymers, semiconductors, carbons, or Li⁺ intercalation materials. Metal nanoparticles include those made from gold or silver. Semi-conductor nanoparticles include those made from silicon or germanium. Polymer nanoparticles include those made from biocompatible or biodegradable polymers. The ability to make nanoparticles from a wide variety of materials or combination of materials allows the creation of nanoparticles with desired biochemical properties such as biocompatibility, including immunogenic compatibility, and/or, biodegradability. In comparison, certain biological delivery systems, such as viral vectors, can cause significant immunogenic phenomena.

Nanoparticles of the present invention can be synthesized using a template synthesis method. For example, nanoparticles can be synthesized using templates prepared from glass (Tonucci, R. J. et al., Science 258, 783 (1992)), xeolite (Beck, J. S. et al., J. Am. Chem. Soc., 114, 10834 (1992)), and a variety of other materials (Ozin, G. A., Adv. Mater., 4, 612 1992)). Alternatively, nanoparticles can be prepared using a self-assembly process, as described in Wang, Z. L., “Structural Analysis of Self-Assembling Nanocrystal Superlattices,” Adv. Mater., 10(1): 13-30 (1998).

In one embodiment, a nanostructure-based assembly of the invention contains a nanoparticle, which has one or more surfaces functionalized to allow attachment of detectors to the surface. Such “functionalized” nanoparticles have at least one surface modified to allow for directed (also referred to as “vectoring”) delivery and/or controlled release of the volatile marker. In certain embodiments, the nanoparticle is formed with an interior void. Different chemical and/or biochemical functional groups can be applied to the inside and/or outside surfaces of the nanoparticle to enable the attachment of an detector and/or volatile marker on a nanoparticle surface.

In another embodiment, the nanostructure-based assembly contains a nanoparticle formed with an interior void to contain a volatile marker and a detachable end-cap with a detector attached thereto. In the presence of a target biomarker, the detector mechanically detaches the end-cap from the nanoparticle to release the volatile marker for analysis by sensor technology.

In a preferred embodiment, the nanoparticle is in the form of a nanotube that is hollow and has a first open end and a second closed end. A volatile marker is enclosed within the hollow interior of the nanotube. The first open end is blocked with an aptamer-bound end-cap that prevents the unintentional release of the volatile marker located within the hollow interior of the nanotube. Once in the presence of a target biomarker, the aptamer binds with the biomarker and causes the end-cap to dissociate from the nanotube and release the volatile marker.

Upon detecting a target biomarker by the aptamer attached to the end-cap, the volatile marker is released with the uncapping of the nanoparticle. The uncapping mechanism may require the use of energy-bearing biomolecular motors such as, but not limited to, the actin-based system (Dickinson, R. B. and D. L. Purich, “Clamped filament elongation model for actin-based motors,” Biophys J., 82:605-617 (2002)). Once the nanoparticle is uncapped, the released marker can then be detected using sensor technology known in the art including, but not limited to, gas chromatography, electronic noses, spectrophotometers to detect the marker's infrared (IF), ultraviolet (UV), or visible absorbance or fluorescence, or mass spectrometers.

Nanotubes

A number of patents and publications describe nanoparticles in the form of tubes (nanotubes). For example, U.S. Pat. No. 5,482,601 to Ohshima et al. describes a method for producing carbon nanotubes. Other methods for making and using nanotubes include the non-carbon nanotubes of Zettl et al., U.S. Pat. No. 6,063,243, and the functionalized nanotubes of Fisher et al., U.S. Pat. No. 6,203,814.

For nanotubes, synthesis occurs within the membrane pores of a microporous membrane or other solid, as described in Charles R. Martin, “Nanomaterials: A Membrane-Based Synthetic Approach,” Science, 266:1961-1966 (1994), using electrochemical or chemical methods. Depending on the membrane and synthetic method used, the nanotubes may be solid or hollow. Template membrane pore diameters can be varied to produce nanotubes having diameters as small as 5 nm to as large as 100 μm. Likewise, the template membrane thickness can be varied to give nanotubes having a length from as small as 5 nm to as large as 100 μm. Preferably, when the nanotube is intended for in vivo use, the nanotube is of length less than 500 μm and diameter less than 200 nm. Especially preferred nanotubes for in vivo use have a maximum dimension less than 100 nm.

“Track-etch” polymeric or porous alumina membranes can be used in the preparation of nanotubes. Track-etch membranes prepared from polycarbonate and polyester are available from suppliers such as Osmonics (Minnetonka, Minn.) and Whatman (Maidstone, Kent UK). Track-etch membranes contain randomly distributed cylindrical pores of uniform diameter that run through the entire thickness of the membrane. Pore diameters as small as 10 nm are commercially available at pore densities of up to 10⁹ pores per square centimeter.

Porous alumina membranes, which are commercially available from Whatman (Maidstone, Kent UK), are prepared electronically from aluminum metal. Pore diameters as small as 5 nm can be achieved at pore densitites as high as 10¹¹ pores per square centimeter. Membranes can be prepared having the membrane thickness from as small as 100 mm to as large as 100 μm.

Nanotubes can be synthesized such that both ends of the nanotube are open. Alternatively, nanotubes having one or two open end(s) can be synthesized. Solid nanotubes can also be synthesized.

Nanotubes with one closed end can be produced by template synthesis, as described above. For example, nanotubes having one closed end can be prepared by terminating the pores in the alumina template into a non-porous alumina barrier layer prior to removal of the alumina template membrane from the substrate aluminum surface (Hornyak, G. L., et al., “Fabrication, Characterization and Optical Properties of Gold-Nanoparticle/Porous-Alumina Composites: The Non-Scattering Maxwell-Garnett Limit,” J. Phys. Chem. B., 101:1548-1555 (1997)). Generally, the non-porous alumina barrier layer is removed when the alumina membrane is stripped off of the aluminum surface. However, if the template synthesis is completed before removal of the alumina from the aluminum, the bottoms of the nanotubes are closed. Dissolution of the alumina then liberates the nanotubes that are closed at one end and open at the other end.

Suitable end-caps used to block a nanotube opening include, for example, nanoparticles having a diameter slightly larger than the inside diameter of the nanoparticle so as to occlude the open end of the nanoparticle. End-caps are any piece of matter and can be composed of materials that are chemically or physically similar (or dissimilar) to the nanoparticle. The end-cap can be a particle that has a maximum dimension of less than 100 μm. In a preferred embodiment, the end-cap is of a spherical or spheroidal form. However, end-caps of other shapes, including ellipsoidal, cylindrical, and irregular, can also be used.

A suitable end-cap can be attached to a nanotube by covalent bonds. For example, silica nanotubes and particles can be linked by disulphide bonds. Initially, the surface at the ends of silica nanotubes is functionalized with a —SH linker. This can be performed while the nanotubes are still embedded in the pores of the template membrane. This allows activation of the end surface without changing the chemical properties of the outer surface of the nanotubes.

If necessary, the inner surfaces of the nanotubes are protected with, for example, a silane group such as (Me—O)₃—(CH₂)₃—OH. After the protection step, the silica surface layers at the nanotube mouths are removed to expose fresh silica. The freshly-exposed silica will be reacted with the silane, such as (Me—O)₃—Si—(CH₂)₃—SH to attach the requisite —SH linker to the mouths of the nanotubes. The length of the alkyl chain in this silane can be varied to allow placement of the —SH linker any desired distance from the nanotube mouth. These —SH functionalities are then reacted with pyridine disulfide in order to obtain nanotubes with an activated disulfide bond at the nanotube ends.

The surface of the end-cap is then functionalized with the same —SH containing silane used on the mouths of the nanotubes. Hence, nanotubes with an activated disulfide at their mouths and end-caps with an —SH group on their surface are available for linkage through disulfide bond formation.

Other types of covalent bonds, for example amide and ester bonds, can be used to attach an end-cap to the nanotube. Siloxane based linking can also be used. This would be particularly useful when the cap is composed of silica as the silanol sites on the silica surface reacts spontaneously with siloxanes to form a covalent oxygen-silicon bond. For metal based nanotubes or end-caps, thiol linkers can be used for attachment. For example, molecule (Me—O)₃—Si—(CH₂)₃—SH could be attached to a silica nanotube and a gold nanoparticle attached as the end-cap using the —SH end of this molecule. It is well known that such thiols form spontaneous As—S bonds with gold surfaces.

Contemplated end-caps for the invention include nanoparticles that can be electrophoretically placed within the mouths of nanotubes so that the entire mouth of the nanotube is blocked when disulfide bonds are formed between the nanotube and the nanoparticle as described in Miller, S. A. and C. R. Martin, “Electroosmotic Flow in Carbon Nanotube Membranes,” J. Am. Chem. Soc., 123(49):12335-12342 (2001).

For example, a nanotube containing membrane is mounted in a U-tube cell with Platinum electrodes immersed into the buffer solution on either side of the membrane. The —SH-functionalized end-caps are added to the cathode half-cell. The buffer solution is maintained at pH=7 so that a small fraction of the —SH groups on the end-caps are deprotonated. These negatively charged particles are driven into the mouths of the nanotubes electrophoretically by using the Platinum electrodes to pass a constant current through the membrane. Hence, the electrophoretic force causes the end-caps to nestle into the nanotube mouths, where disulfide bond formation will occur.

As an alternative to the electrophoretic assembly method, —SH labeled end-caps can be suspended in solution together with the activated disulfide labeled nanotubes. Here, the nanoparticle caps can spontaneously self-assemble to the nanotubes. The self-assembly of gold nanospheres and latex particles to template prepared polymeric and metal nanowires is described by Sapp, S. A. et al., “Using Template-Synthesized Micro- and Nanowires as Building Blocks for Self-Assembly of Supramolecular Architectures,” Chem. Mater., 11:1183-1185 (1999).

In addition to —SH linking, other covalent linking methods can be used to link nanotubes and end-caps. Non-covalent linking methods can be used. These include, for example, DNA hybridization (Mirkin, C. A., “Programming the Self-Assembly of Two and Three-Dimensional Architectures with DNA and Nanoscale Inorganic Building Blocks,” Inorg. Chem., 39:2258-2272 (2000)), the biotin/avidin interaction (Connolly, S. and D. Fitzmaurice, “Programmed Assembly of Gold Nanocrystals in Aqueous Solution,” Adv. Mater., 11:1202-1205 (1999)), and antigen/antibody interactions (Shenton, W. et al., “Directed Self-Assembly of Nanoparticles into Macroscopic Materials Using Antibody-Antigen Recognition,” Adv. Mater., 11:449 (1999)).

Preferred nanotubes are those comprising silica or polymers. Silica nanotubes can be prepared using sol-gel template synthesis, as described in Lakshmi, B. B. et al., “Sol-Gel Template Synthesis of Semiconductor Oxide Micro- and Nanostructures,” Chem. Mater., 9:2544-2550 (1997); Lakshmi, B. B. et al., “Sol-Gel Template Synthesis of Semiconductor Nanostructures,” Chem Mater., 9:857-862 (1997). The template membrane is immersed into a standard tetraethylorthosilicate sol so that the sol fills the pores. After the desired emersion time, the membrane is removed, dried in air, and then cured at 150° C. This yields silica nanotubes lining the pore walls of the membrane plus silica surface films on both faces of the membrane. The surface films are removed by briefly polishing with slurry of alumina particles. The nanotubes are then liberated by dissolving the template membrane and collected by filtration.

The outside diameter of the nanotube can be controlled by varying the pore diameter of the template membrane, the length of the nanotube can be controlled by varying the thickness of the template membranes, and the inside diameter of the nanotube can be controlled by varying the immersion time in the sol.

Polymer nanotubes can be prepared from many substances that are composed of monomer units. “Monomer units,” as used herein, refers to the individual moieties that are repeated to form “polymers.” Multiple monomer units are covalently attached when tin the form of a backbone of a polymer. Polymers that are made from at least two different types of monomer units are referred to as “copolymers.” Polymerizing or copolymerizing describes the process by which multiple monomers are reacted to form covalently linked monomer units that form polymers or copolymers, respectively. A discussion of polymers, monomer units, and the monomers from which they are made may be found in Stevens, Polymer Chemistry: An Invitation, 3^{rd} ed., Oxford University Press (1999).

Polymeric nanotubes can be prepared using a solution deposition method as described in Depak, V. M. and C. R. Martin, “Preparation of Polymeric Micro- and Nanostructures Using a Template-Based Deposition Method,” Chem. Mater., 11:1363-1367 (1999). This method entails depositing a solution of the desired polymer within the pores of the template membrane and allowing the solvent to evaporate. In addition, polymer nanotubes can be prepared by polymerizing a monomer of a monomer within the pore as described by Martin, C. R., “Template Synthesis of Electronically Conductive Polymer Nanostructures,” Acc. Chem. Res., 28:61-68 (1995).

Preferred polymers include polystyrene, polyorganosiloxane, poly(methyl methacrylate), polystyrene, polylactic acids, and other biodegradable polymers, acrylic latexes, polyorganosiloxane, cellulose, polyethylene, poly(vinyl chloride), poly(ethyl methacrylate), poly(tetrafluoroethylene), poly(4-iodostyrene/divinylbenzene), poly(4-vinylpyridine/divinylbenzene), poly(styrene/divinyl benzene), crosslinked melamine particles, phenolic polymer colloids, polyamide 6/6, natural rubber, naturally occurring biopolymers such as algenates, and collagen, or mixtures thereof.

When the nanotubes are to be introduced into a patient, for example, when used as a nanostructure-based assembly for the detection and notification of a disease or medical condition, biodegradable polymers and biocompatible polymers are especially preferred. Examples of useful biodegradable polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

Preferably, biocompatible are characterized by immunogenic compatibility. Examples of biocompatible polymers that can be used in the manufacture of nanotubes of the invention include PLG [Poly(lactide-co-glycolide)], poly(ethylene glycol), copolymers of poly(ethylene oxide) with poly(L-Lactic acid) or with poly(β-benzyl-L-aspartate. In addition, a number of approaches can be used to make a nanotube surface biocompatible and “stealthy.” For example, this can be accomplished by attaching a PEG-maleimide to the chain-end thiols on the outer surfaces of the nanotube. If the nanotube is composed of Au or similar metals, the PEG chain can be attached by a thiol linker as described in Yu, S.; Lee, S. B.: Kang, M.: Martin, C. R. “Size-Based Protein Separations in Poly(ethylene glycol)—Derivatized Gold Nanotubule Membranes,” Nano Letters, 1:495-498 (2001). Other examples of biocompatible polymers and surface treatments can be found in Majeti N. V. Ravi Kumar, “Nano and Microparticles as Controlled Drug Delivery Devices” J. Pharm. Pharmaceut. Sci. 3(2): 234-258 (2000), the contents of which are incorporated by this reference.

In one embodiment of the invention, a nanostructure-based assembly includes a nanotube with a hollow interior comprising a volatile marker. The nanotube is constructed using known methods such as those disclosed in U.S. patent application Ser. No. 10/274,829, filed Oct. 21, 2002. The nanotube further includes a detector for localizing the nanostructure-based assembly to a target biomarker. The volatile marker is released from the nanostructure-based assembly when in the presence of a target biomarker.

In a related embodiment, release of the volatile marker from the hollow void is achieved by “uncapping” the nanotube. An end-cap is placed over an opening to the void to function as a means for controlling the release of the contents therein (i.e., volatile marker). Methods for attaching an end-cap to a nanoparticle include, but are not limited to, using: electrostatic attraction, hydrogen bonding, acid and/or basic sites located on the end-cap/nanoparticle, covalent bonds, and other chemical linkages.

In a preferred embodiment, the detecting means is attached to the end-cap to affect the release of the volatile marker via uncapping of the nanoparticle. For example, the uncapping mechanism is based upon the detection by the detecting means of certain biomarkers including for example, surface markers on cell types (i.e., reticulocytes), proteins in the blood (i.e., vitamin K epoxide reductase), or drugs in the body (i.e., warfarin). The uncapping mechanism may require the use of energy-bearing biomolecular motors such as, but not limited to, the actin-based system (Dickinson, R. B. and D. L. Purich, “Clamped filament elongation model for actin-based motors,” Biophys J., 82:605-617 (2002)).

The released volatile marker can then be detected using sensor technology known in the art including, but not limited to, gas chromatography, electronic noses, spectrophotometers to detect the detectable marker's infrared (IF), ultraviolet (UV), or visible absorbance or fluorescence, or mass spectrometers.

Functionalization of the Nanoparticles

According to the present invention, nanoparticles can be prepared having different chemically or biochemically functionalized surfaces to enable attachment of a detector and/or volatile marker. Methods used to functionalize a nanoparticle surface depend on the composition of the nanoparticle and are well known in the art. For example, functionalization of silica nanoparticles is accomplished using silane chemistry. With silane chemistry, different functional groups can be attached to the surfaces of the nanoparticle by attaching a functional group to the nanoparticle surface while the nanoparticles are embedded within the pores of the template. Then, a hydrolytically unstable silane is reacted with the surface silanol sites on the nanoparticle to obtain covalent oxygen/silicon bonds between the surface and the silane. Additional functional groups can also be attached to the nanoparticle surface after dissolution of the template.

The surface of polymer nanoparticles can also be functionalized using well known chemical methods. For example, methods employed for polylactide synthesis allow for differential end-functionalization. Polymerization occurs by an insertion mechanism mediated by Lewis acids such as Sn²⁺ whose bonds with oxygen have significant covalent character. An alcohol complexed with the metal ion initiates polymerization, which continues by stepwise ring-opening of the lactide monomers to generate a new alkoxide-metal complex capable of chain growth. The polymer molecular weight can be controlled by the molar ratio of initiating alcohol to the lactide monomer. The resulting polyester possesses directionality with a hydroxyl terminus (from the first monomer) and a functional group at the ester terminus determined by the structure of the initiating alcohol. The latter can contain a variety of functional groups to enable attachment of a detecting means, marker, and/or payload to a nanoparticle surface.

Alternatively, functional groups can be introduced by copolymerization. Natural amino acids are sterically similar to lactic acid but offer a variety of functional groups on their side chains (—OH, —CO₂H, —NH₂, —SH, etc.). Moreover, amino acids are found in all cell types, so that the polymer degradation products are non-toxic. Monomers derived from an amino acid and lactic acid can be synthesized by standard methods and used for random copolymerization with lactide. In accordance with the present invention, nanoparticles can have functional groups on any surface to enable the attachment of a detector and/or a volatile marker. Such functional groups allow the nanostructure-based assembly to be bioengineered to accomplish specific functions, such as detect, provide notification of, and treat specific diseases.

In addition, the detecting means and/or volatile marker can be incorporated into the nanoparticle framework, which can include chitosan, PEGylated PLGA (poly(lactic-co-glycolic acid), or other PEGylated compounds. For example, a commercially available PEG-maleimide can be incorporated into chain-end thiols on the outer surface of the nanoparticles. Alternatively, the detecting means and/or volatile marker can be incorporated into nanoparticle frameworks composed of biodegradable and/or resorbable materials including, for example, polylactide based polymers as described above.

For nanoparticles comprising a hollow void in which the volatile marker can be contained, a volatile marker can be loaded into the void using an electrophoretic force. (See Miller, S. A. and C. R. Martin, “Electroosmotic Flow in Carbon Nanotube Membranes,” J. Am. Chem. Soc., 123(49):12335-12342 (2001)). Alternatively, nanoparticles embedded within the synthesis membrane can be filled with a volatile marker by vacuum filtering a solution containing the volatile marker through the synthesis membrane. (See Parthasarathy, R. and C. R. Martin, Nature, 369:298 (1994)). For nanoparticles prepared by formation within an alumina template film prior to removal of the alumina from the underlying aluminum surface, they can be filled by simply applying a solution containing the volatile marker to the surface of the film (where the opening to the hollow void is located) and allowing the solvent to evaporate. Multiple applications can be used, if needed.

In one embodiment, a detector is attached to the outer surface of the nanoparticle via any of the aforementioned functional groups. The controlled release of the volatile marker is accomplished by the release of the end-cap, which is attached to the nanoparticle via chemically labile bonds.

Yet another embodiment provides a nanoparticle that has the detecting means, the volatile marker applied to the outside, exposed surface of the nanoparticle. These components are attached to the surface of the nanoparticle via chemically labile bonds, which allow for the release of these components under specific conditions (such as release of a volatile marker after detector identification of a biomarker).

In a related embodiment, aptamers are used as detectors. Aptamers can be attached to proteins utilizing methods well known in the art (see Brody, E. N. and L. Gold, “Aptamers as therapeutic and diagnostic agents,” J Biotechnol, 74(1):5-13 (2000) and Brody, E. N. et al., “The use of aptamers in large arrays for molecular diagnostics,” Mol Diagn, 4(4):381-8 (1999)). For example, photo-cross-linkable aptamers allow for the covalent attachment of aptamers to proteins. Such aptamer-linked proteins can then be immobilized on a functionalized surface of a nanoparticle.

For example, aptamer-linked proteins can be attached covalently to a nanoparticle end-cap or to an exterior nanoparticle surface, including attachment of the aptamer-linked protein by functionalization of the surface. Alternatively, aptamer-linked proteins can be covalently attached to a nanoparticle surface via linker molecules. Non-covalent linkage provides another method for introducing aptamer-linked proteins to a nanoparticle surface. For example, an aptamer-linked protein may be attached to an nanoparticle surface by absorption via hydrophilic binding or Van der Waals forces, hydrogen bonding, acid/base interactions, and electrostatic forces.

An identification of a biomarker by the detector affects the release of the volatile marker from the nanoparticle. Because the volatile marker is released from the nanoparticle only in the presence of a biomarker, detection of the volatile marker in a bodily fluid sample provides notice that the biomarker is present in the patient and consequently, allows diagnosis of the specific disease associated with the biomarker.

In certain embodiments, the nanostructure-based assemblies of the invention have the ability to differentiate between and provide a signal regarding different types of blood cells and their concentration in the patient. For example, levels of red blood cells (RBCs), white blood cells (WBCs), and platelets can be assessed using the systems and methods of the invention to diagnose hematopoiesis abnormalities such as leukemia or assess changes in cellular content (for example, RBC content). Accordingly, the subject invention is useful in diagnosing blood-based diseases or disorders including, without limitation, hemorrhagic diathesis (i.e., hemophilia, von Willebrand disease, Alexander's disease, Telfer's disease, Owren's parahemophilia, prothrombin deficiency); non-hemorrhagiparous coagulopathies (i.e., Fletcher factor deficiency, Flaujeac factor deficiency); thrombophilic coagulopathies (i.e., Ratnoff's disease, thrombomodulin deficiency); thrombocytopenia; anemias; and alterations in white blood cells (i.e., Pelger-Huet anomaly (PHA); Chediak-Higashi syndrome (CHS); Hegglin-May anomaly (HMA)).

Detectors

A nanostructure-based assembly of the invention comprises a nanoparticle, which contains a means for detecting a target biomarker and a volatile marker. In a preferred embodiment, a detector is designed to detect a target biomarker.

The detector of the invention can be selected from biosensors known to the skilled artisan. Such biosensors include naturally occurring and/or synthetic compounds having high specificity and sensitivity to chemical and/or biological compounds of interest. Suitable biosensors of the invention include, but are not limited to, antibodies, proteins, and aptamers.

Certain detectors of the invention are designed to alter the biological function of the target biomarker. Other detectors can be designed to localize nanostructure-based assemblies within the vicinity of or into target cells for optimal release a volatile marker. In related embodiments, the detecting means also has the capability of cellular localization (i.e., delivering the nanostructure-based assembly to a cancer cell) or subcellular localization (i.e., delivering the nanostructure-based assembly to a nucleus within a cancer cell).

The detecting means of the invention can allow for applications requiring specific biomarker localization or immobilization (i.e., vectoring). See Langer, R., “Tissue Engineering,” Mol Ther, 2:12-15 (2000). Detecting means including, for example, proteins, antibodies, peptides, RNA or DNA aptamers, cellular reporters or cellular ligands, can be attached to a nanoparticle surface to provide a means for vectoring the nanostructure-based assembly to a target biomarker. Such detectors may be covalently attached to the nanoparticle surface. In certain related embodiments, detectors are attached to a nanoparticle surface via linker molecules. Detectors can also be attached to a nanoparticle surface by non-covalent linkage, for example, by absorption via hydrophobic binding or Van der Waals forces, hydrogen bonding, acid/base interactions, and electrostatic forces.

In one embodiment, the detector is an antibody specific to a target biomarker. The antibody has a recognized structure that includes an immunoglobulin heavy and light chain. The heavy and light chains include an N-terminal variable region (V) and a C-terminal constant region (C). The heavy chain variable region is often referred to as “V_{H}” and the light chain variable region is referred to as “V_{L}”. The V_{H} and V_{L} chains form a binding pocket that has been referred to as F(v). See generally Davis, 3: 537, Ann. Rev. of Immunology (1985); and Fundamental Immunology 3rd Ed., W. Paul Ed. Raven Press LTD. New York (1993). The shape of the binding pocket F(v) is complementary to that of the target biomarker for efficient and specific binding. Generally, binding is due to weak, non-covalent bonds between the antibody and that of the target biomarker. Antibodies can be attached to the nanoparticle using methods known to the skilled artisan.

Alternatively, recombinant bispecific antibody (bsFv) molecules can be used as a detector. In a preferred embodiment, bsFv molecules that bind a T-cell protein termed “CD3” and a TAA are used as a detector in accordance with the present invention. In related embodiments, bsFv molecules are used not only to specifically bind to a target biomarker but also to facilitate an immune system response. See Jost, C. R. 33: 211, Mol. Immunol (1996); Lindhofer, H. et al. 88: 465 1, Blood (1996); Chapoval, A. I. et al. 4: 571, J. of Hematotherapy (1995).

With other embodiments of the present invention, the detector is in the form of an aptamer. Aptamers have the ability to recognize a broad range of targets, including small organic molecules as well as large proteins (Gold et al., supra.; Osborne and Ellington, “Nucleic acid selection and the challenge of combinatorial chemistry,” Chem. Rev., 97:349-370 (1997)).

Aptamers derived from the SELEX™ methodology can be utilized in the present invention. The SELEX™ methodology enables the production of aptamers, each of which have a unique sequence and the property of binding specifically to a desired target compound or molecule. The SELEX™ methodology is based on the insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. See also Jayasena, S., “Aptamers: An Emerging Class of Molecules That Rival Antibodies for Diagnostics,” Clinical Chemistry, 45:9, 1628-1650 (1999).

Aptamers that can be used as detectors in the present invention include those described in U.S. Pat. No. 5,656,739 (hereinafter the '739 patent), which discloses the advantages of synthetic oligonucleotides as assembly templates. The '739 patent describes nucleic acids as particularly useful assembly templates because they can be selected to specifically bind nonoligonucleotide target molecules with high affinity (e.g., Tuerk and Gold (1990), supra), and because they can hybridize by complementary base pairing. Both forms of recognition can be programmably synthesized in a single molecule or hybridized into a single discrete structure.

Volatile Markers

The volatile marker of the invention can be any compound that can be identified in bodily fluids including radio-labeled or fluorescent compounds, compounds that change the color of bodily fluids for detection by the naked eye, or compounds that are readily identified in bodily fluids using sensor technology. Preferably, the detection of a volatile marker in bodily fluids indicates the presence of target biomarkers in the patient and enables the diagnosis of the disease associated with the target biomarker. In certain embodiments, the volatile marker is the biomarker itself.

The volatile marker of the invention can be a marker (such as benzodiazepine, benzodiazepine metabolite, phenobarbital, or phenobarbital metabolite) that is detectable in urine. For example, benzodiazepines and their metabolites readily pass through the renal system into urine making benzodiazepines and substances with similar properties especially suitable as compliance markers. Examples of benzodiazepines or benzodiazepine metabolites that can be used in the invention include diazepam and alprazolam.

Additional volatile markers contemplated herein include, without limitation, dimethyl sulfoxide (DMSO), acetaldehyde, acetophenone, anise, benzaldehyde, benzyl alcohol, benzyl cinnamate, cadinene, camphene, camphor, cinnamon, garlic, citronellal, cresol, cyclohexane, eucalyptol, and eugenol, eugenyl methyl ether. Such markers are particularly advantageous for use in detection in exhaled breath.

The volatile markers of the invention also include additives that have been federally approved and categorized as GRAS (“generally recognized as safe”), which are available on a database maintained by the U.S. Food and Drug Administration Center for Food Safety and Applied Nutrition. Surrogate markers categorized as GRAS and are readily detectable in bodily fluids include, and are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

As described above, volatile markers are detected by their physical and/or chemical properties, which does not preclude using a target biomarker as its own volatile marker.

Sensor Technology

Sensor technology is used by the present invention to detect the presence of a volatile marker in a bodily fluid sample. The detection of a volatile marker signifies the presence and/or quantity of a target biomarker.

A sensor of the invention can be selected from a wide variety of sensor technology including, but is not limited to, those described in U.S. Pat. Nos. 6,010,459; 5,081,871; 5,042,501; 4,202,352; 5,971,937, and 4,734,777, “artificial” or electronic” noses or tongues, semiconductor gas sensor technology, conductive polymer gas sensor technology, surface acoustic wave gas sensor technology, immunoassays, metal-insulator-metal ensemble (MIME) sensors, cross-reactive optical microsensor arrays, fluorescent polymer films, and surface enhanced raman spectroscopy (SERS).

The present invention contemplates using sensor technology based on surface acoustic wave (SAW) sensors. These sensors oscillate at high frequencies and respond to perturbations proportional to the mass load of certain molecules. This occurs in the vapor phase on the sensor surface. The resulting frequency shift is detected and measured by a computer. Usually, an array of sensors (such as 4-6 sensors) is used, each coated with a different chemoselective polymer that selectively binds and/or absorbs vapors of specific classes of molecules. The resulting array, or “signature” identifies specific compounds. Sensitivity of the arrays is dependent upon the homogeneity and thickness of the polymer coating.

Surface-acoustic-wave (SAW) gas-sensors generally include a substrate with piezoelectric characteristics covered by a polymer coating, which is able to selectively absorb a volatile marker. The variation of the resulting mass leads to a variation of its resonant frequency. This type of sensor provides very good mass-volume measures of the volatile markers. In the SAW device, the volatile marker is used to propagate a surface acoustic wave between sets of interdigitated electrodes. The chemoselective material is coated on the surface of the transducer. When a volatile marker interacts with the chemoselective material coated on the substrate, the interaction results in a change in the SAW properties, such as the amplitude or velocity of the propagated wave. The detectable change in the characteristics of the wave indicates the presence and concentration of the volatile marker (and corresponding target biomarker).

A SAW vapor sensing device has been disclosed in which a layer of antibodies are attached to a surface of the SAW sensor (see Stubbs, D D et al., “Investigation of Cocaine Plumes Using Surface Acoustic Wave Immunoassay Sensors,” Anal. Chem., 75:6231-6235 (2003)). When a target antigen reacts with an antibody, the acoustic velocity is altered, causing an oscillator frequency of the SAW to shift to a different value. The subject invention contemplates usage of such SAW devices, as well as those SAW sensing devices in which aptamers (including indicator aptamers), molecular beacons, and other known biosensors are utilized to coat a surface of the SAW sensor.

Certain embodiments use known SAW devices described in numerous patents and publications, including U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator adsorbent,” Analytica Chimica Acta, 371:131-143 (1988).

Other types of chemical sensors known in the art that use chemoselective coating applicable to the operation of the present invention include bulk acoustic wave (BAW) devices, plate acoustic wave devices, interdigitated microelectrode (IME) devices, optical waveguide (OW) devices, electrochemical sensors, and electrically conducting sensors.

In another embodiment, the invention uses fluid sensor technology, such as commercial devices known as “artificial noses,” “electronic noses,” or “electronic tongues.” These devices are capable of qualitative and/or quantitative analysis of simple or complex gases, vapors, odors, liquids, or solutions. A number of patents and patent applications which describe fluid sensor technology include the following: U.S. Pat. Nos. 5,945,069; 5,918,257; 5,891,398; 5,830,412; 5,783,154; 5,756,879; 5,605,612; 5,252,292; 5,145,645; 5,071,770; 5,034,192; 4,938,928; and 4,992,244; and U.S. Patent Application No. 2001/0050228. Certain sensitive, commercial off-the-shelf electronic noses, such as those provided by Cyrano Sciences, Inc. (“CSI”) (i.e., CSI's portable Electronic Nose and CSI's Nose-Chip™ integrated circuit for odor-sensing—U.S. Pat. No. 5,945,069), are preferably used in the present invention to detect the presence of detectable volatile markers in bodily fluid samples.

Other embodiments of the present invention use sensor technology selected from semiconductive gas sensors; mass spectrometers; and IR, UV, visible, or fluorescence spectrophotometers. With these sensors, a volatile marker changes the electrical properties of the semiconductors by making their electrical resistance vary, and the measurement of these alternatives allows the determination of the concentration of detectable markers present in the sample. The methods and apparatus used for detecting markers generally have a brief detection time of a few seconds.

Additional recent sensor technologies included in the present invention include apparatus having conductive-polymer gas-sensors (“polymeric”), aptamer biosensors, and amplifying fluorescent polymer (AFP) sensors.

Conductive-polymer gas-sensors (also referred to as “chemoresistors”) are coated with a film sensitive to the molecules of certain detectable markers. On contact with the molecules, the electric resistance of the sensors change and the measurement of the variation of this resistance enable the concentration of the detected substance (i.e., volatile marker and corresponding target biomarker in blood) to be determined. An advantage of this type of sensor is that it functions at temperatures close to ambient. Different sensitivities for detecting different detectable markers can be obtained by modifying or choosing an alternate conductive polymer.

Polymeric gas sensors can be built into an array of sensors, where each sensor responds to different gases and augment the selectivity of the volatile marker.

Aptamer-based biosensors can be utilized in the present invention for detecting the presence of volatile markers in bodily fluid samples. Aptamer biosensors are resonant oscillating quartz sensors that can detect minute changes in resonance frequencies due to modulations of mass of the oscillating system, which results from a binding or dissociation event.

Similarly, amplifying fluorescent polymer (AFP) sensors may be utilized in the present invention for detecting the presence of detectable markers in bodily fluid samples. AFP sensors are extremely sensitive and highly selective chemosensors that use amplifying fluorescent polymers. When vapors bind to thin films of the polymers, the fluorescence of the film decreases. A single molecule binding event quenches the fluorescence of many polymer repeat units, resulting in an amplification of the quenching. The binding of target markers to the film is reversible, therefore the films can be reused.

In accordance with the present invention, competitive binding immunoassays can be used to test a bodily fluid sample for the presence of volatile markers. Immunoassay tests generally include an absorbent, fibrous strip having one or more reagents incorporated at specific zones on the strip. The bodily fluid sample is deposited on the strip and by capillary action the sample will migrate along the strip, entering specific reagent zones in which a chemical reaction may take place. At least one reagent is included which manifests a detectable response, for example a color change, in the presence of a minimal amount of a marker of interest. Patents that describe immunoassay technology include the following: U.S. Pat. Nos. 5,262,333 and 5,573,955.

Other embodiments of the present invention use flow cytometers to analyze bodily fluid samples for target compounds. Flow cytometry is a technique that is used to determine certain physical and chemical properties of microscopic biological particles by sensing certain optical properties of the particles. To do so, the particles are arranged in single file using hydrodynamic focusing within a sheath fluid. The particles are then individually interrogated by a light beam. Each particle scatters the light beam and produces a scatter profile. The scatter profile is often identified by measuring the light intensity at different scatter angles. Certain physical and/or chemical properties of each particle can then be determined from the scatter profile. Patents that describe flow cytometry technology include the following: U.S. Pat. Nos. 6,597,438; 6,097,485; 6,007,775; and 5,716,852.

The sensor of the present invention might include integrated circuits (chips) manufactured in a modified vacuum chamber for Pulsed Laser Deposition of polymer coatings. It will operate the simultaneous thin-film deposition wave detection and obtain optimum conditions for high sensitivity of SAW sensors. The morphology and microstructure of biosensor coatings will be characterized as a function of process parameters.

Where the bodily fluid is exhaled breath, the sensor used in the subject invention may be modified so that patients can exhale directly onto the sensor, without needing a breath sampling apparatus. For example, a mouthpiece or nosepiece will be provided for interfacing a patient with the device to readily transmit the exhaled breath to the sensor (See, i.e., U.S. Pat. No. 5,042,501). In a related embodiment, wherein the sensor is connected to a neural network, the output from the neural network is similar when the same patient exhales directly into the device and when the exhaled gases are allowed to dry before the sensor samples them.

The humidity in the exhaled gases represents a problem for certain electronic nose devices (albeit not SAW sensors) that only work with “dry” gases. When using such humidity sensitive devices, the present invention may adapt such electronic nose technology so that a patient can exhale directly into the device with a means to dehumidify the samples. This is accomplished by including a commercial dehumidifier or a heat moisture exchanger (HME), a device designed to prevent desiccation of the airway during ventilation with dry gases.

Alternatively, the patient may exhale through their nose, which is an anatomical, physiological dehumidifier to prevent dehydration during normal respiration. Alternatively, the sensor device can be fitted with a preconcentrator, which has some of the properties of a GC column. The gas sample is routed through the preconcentrator before being passed over the sensor array. By heating and volatilizing the gases, humidity is removed and the marker being measured can be separated from potential interferents.

Preferably, in operation, the sensor will be used to identify a baseline spectrum for the patient prior to drug administration, if necessary. This will prove beneficial for the detection of more than one therapeutic drug if the patient receives more than one drug at a time and possible interference from different foods and odors in the stomach, mouth, esophagus and lungs.

Applications

Specific hematological conditions/diseases that can be detected using a sensor according to the present invention are listed in Merck Manual Diagnosis and Therapy, 17^{th} ed., Merck & Company, Inc., 1999, which include, but are not limited to, Afibrinogenemia, Agammaglobulinemia, Anemia (i.e., aplastic anemia, hemolytic anemia, congenital nonspherocytic anemia, megaloblastic anemia, pernicious anemia, sickle cell anemia, Fanconi Anemia), Angiolymphoid Hyperplasia with Eosinophilia, Antithrombin III Deficiency, Bernard-Soulier Syndrome, Blue Rubber Bleb Nevus Syndrome, Chediak-Higashi Syndrome, Cryoglobulinemia, Disseminated Intravascular Coagulation, Eosinophilia, Erdheim-Chester Disease, Erythroblastosis, Fetal—Evans Syndrome, Factor V Deficiency, Factor VII Deficiency, Factor X Deficiency, Factor XI Deficiency, Factor XII Deficiency, Giant Lymph Node Hyperplasia, Hemoglobinuria, Paroxysmal—Hemoglobinuria, Paroxysmal—Hemophilia A, Hemophilia B, Hemorrhagic Disease of Newborn, Histiocytosis, Langerhans-Cell, Non-Langerhans-Cell, Job's Syndrome, Leukopenia, Lymphadenitis, Lymphangiomyomatosis, Lymphedema, Methemoglobinemia, Myelodysplastic Syndromes, Myelofibrosis, Myeloid Metaplasia, Myeloproliferative Disorders, Neutropenia, Paraproteinemias, Platelet Storage Pool Deficiency, Polycythemia Vera, Protein C Deficiency, Protein S Deficiency, Purpura (i.e., thrombocytopenic purpura, thrombotic thrombocytopenic purpura), Sarcoidosis, Spherocytosis, Splenic Rupture, Thalassemia, Thrombasthenia, Thrombocytopenia, Waldenstrom Macroglobulinemia, and von Willebrand Disease.

Reporting Means and Data Monitor/Analyzer

Results from sensor technology analysis of bodily fluid samples are optionally provided to the user (or patient) via a reporting means. In one embodiment, sensor technology includes the reporting means. Contemplated reporting means include a computer processor linked to the sensor technology in which electronic or printed results can be provided. Alternatively, the reporting means can include a digital display panel, transportable read/write magnetic media such as computer disks and tapes_which can be transported to and read on another machine, and printers such as thermal, laser or ink-jet printers for the production of a printed report.

The reporting means can provide the results to the user (or patient) via facsimile, electronic mail, mail or courier service, or any other means of safely and securely sending the report to the patient. Interactive reporting means are also contemplated by the present invention, such as an interactive voice response system, interactive computer-based reporting system, interactive telephone touch-tone system, or other similar system. The report provided to the user (or patient) may take many forms, including a summary of analyses performed over a particular period of time or detailed information regarding a particular bodily fluid sample analysis. Results may also be used to populate a financial database for billing the patient, or for populating a laboratory database or a statistical database.

A data monitor/analyzer can compare a pattern of response to previously measured and characterized responses from known markers. The matching of those patterns can be performed using a number of techniques, including neural networks. By comparing the analog output from each of the 32 polymers to a “blank” or control, for example, a neural network can establish a pattern that is unique to that marker and subsequently learns to recognize that marker. The particular resistor geometries are selected to optimize the desired response to the target marker being sensed. The sensor of the subject invention is preferably a self-calibrating polymer system suitable for detecting and quantifying markers in gas phase biological solutions to assess and/or monitor a variety of therapeutic drug markers simultaneously.

According to the subject invention, sensor technology can include a computer that communicates therewith, which can also notify the medical staff and/or the patient as to the medical condition of the patient. In certain embodiments, the subject system enables determination as to whether a patient has been administered a pharmacologically effective amount of a therapeutic drug (i.e., warfarin for anticoagulation). The device could also alert the patient (or user) as to time intervals and/or dosage of therapeutic drug to be administered. Accordingly, it is contemplated herein that a sensor of the subject invention can be portable.

In certain embodiments, the subject invention enables the immediate monitoring of levels specific cellular components associated with a therapeutic drug in a patient's blood stream. As contemplated herein, immediate monitoring refers to sampling and analysis of exhaled gases from a patient for target markers substantially completely within a short time period following administration of a therapeutic drug (i.e., generally within a few minutes to about 24 hours).

Alternatively, in certain instances, a specific period of time must progress before the concentration of a cellular component associated with a therapeutic drug in the blood stream can be detected. Accordingly, a system and/or method of the invention can be provided to a patient taking a therapeutic drug for intermittent or continuous monitoring of therapeutic drug concentrations in the blood stream. In certain embodiments, the monitoring system and method of the subject invention can be administered to a patient taking a therapeutic drug on an hourly, daily, weekly, monthly, or even annual basis. Further, additional monitoring can be administered to a patient when an additional therapeutic drug is prescribed.

Moreover, a CPU may be provided as a data processing/control unit for automatically detecting a signal from a sensor of the invention to control sampling of exhaled breath. The CPU may further provide to the user/patient the appropriate dosage of the therapeutic drug to be delivered based on analysis of trends in therapeutic drug blood concentration.

Depending on the mode of therapeutic drug administration, the present invention provides means for automatically adjusting and administering the appropriate dosage of a therapeutic drug, based on blood concentration levels, to a patient. In certain embodiments, a CPU is provided for analysis and control of dosage adjusting and administering means. In one embodiment in which a therapeutic drug is delivered intravenously, an infusion pump is used, wherein the CPU provides analysis and control of the infusion pump.

Bodily Fluid Samples

According to the subject invention, a sample of bodily fluid (such as blood or exhaled breath) is collected from the patient for analysis via sensor technology. The bodily fluid sample is analyzed for the presence of volatile markers associated with cellular components of interest. Detection of the volatile marker indicates the presence of the target cellular component in the patient and consequently, allows for the non-invasive, point-of-care disease diagnosis.

Generally, the exhalation gas stream comprises sequences or stages. At the beginning of exhalation there is an initial stage, the gas representative thereof coming from an anatomically inactive (deadspace) part of the respiratory system, in other words, from the mouth and upper respiratory tracts. This is followed by a plateau stage. Early in the plateau stage, the gas is a mixture of deadspace and metabolically active gases. The last portion of the exhaled breath comprises nothing but deep lung gas, so-called alveolar gas. This gas, which comes from the alveoli, is termed end-tidal gas.

In a preferred embodiment, the exhaled breath sample is collected at end-tidal breathing. Technology similar to that used for end-tidal carbon dioxide monitoring can be used to determine when the sample is collected. Known methods for airway pressure measurements afford another means of collecting samples at the appropriate phase of the respiratory cycle. Single or multiple samples collected by the known side stream method are preferable, but if sensor acquisition time is reduced, in-line sampling may be used. In the former, samples are collected through an adapter at the proximal end of an endotracheal (ET) tube and drawn through thin bore tubing to a sensor of the subject invention.

Depending on the sample size and sensor response time, exhaled gas may be collected on successive cycles. With in-line sampling, a sensor of the subject invention is placed proximal to the ET tube directly in the gas stream. Alternatively to sample end-tidal gas, samples can be taken throughout the exhalation phase of respiration and an average value determined and correlated with blood concentration.

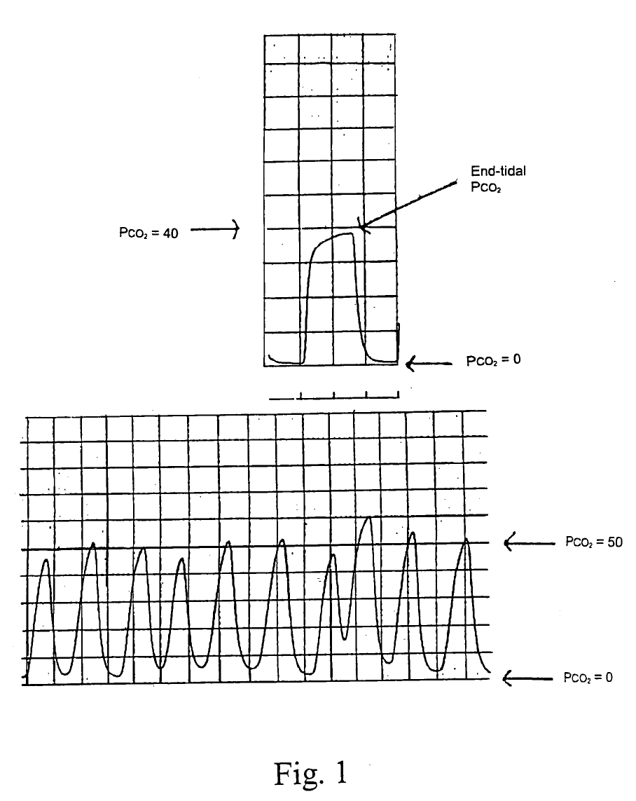

Referring now to FIG. 1, the upper frame demonstrates a capnogram of a single respiratory cycle. For accurate blood level correlation, samples are taken at the point labeled “end-tidal PCO₂” which reflects the CO₂ concentration in the lung. The lower frame shows a capnogram of several breaths from a patient with obstructive lung disease. Again the end-tidal sample correlated best with blood concentration.

In one embodiment, a VaporLab™ brand instrument is used to collect and analyze exhaled breath samples. The VaporLab™ instrument is a hand-held, battery powered SAW-based chemical vapor identification instrument suitable for detecting components in exhaled breath samples in accordance with the present invention. This instrument is sensitive to volatile and semi-volatile compounds using a high-stability SAW sensor array that provides orthogonal vapor responses for greater accuracy and discrimination. In a related embodiment, this instrument communicates with computers to provide enhanced pattern analysis and report generation. In a preferred embodiment, this instrument includes neural networks for “training” purposes, i.e., to remember chemical vapor signature patterns for fast, “on-the-fly” analysis.

In another embodiment, samples are collected at the distal end of an ET tube through a tube with a separate sampling port. This may improve sampling by allowing a larger sample during each respiratory cycle.

In certain instances, the concentration of a therapeutic drug in a patient body is regulated by the amount of the drug administered over a given time period and the rate at which the agent is eliminated from the body (metabolism). The present invention provides the steps of administering a therapeutic drug to a patient and analyzing patient exhaled breath for concentration of therapeutic drug markers such as unbound substances, active metabolites, or inactive metabolites associated with the therapeutic drug, after a suitable time period. In certain embodiments of the subject invention, the marker concentration indicates a characteristic of metabolism of the drug in the patient.

Methods of the subject invention may further include the use of a flow sensor to detect starting and completion of exhalation. The method further includes providing and communicating the results from the analysis. In a preferred embodiment, results from analysis can be communicated immediately upon sampling exhaled gases. Moreover, a CPU may be provided as a data processing/control unit for automatically detecting the signal from the flow sensor to control sampling of exhaled breath. The CPU may further provide to the user/patient health diagnosis based on analysis of trends in blood component(s) concentration.

In another embodiment, the exhalation air is measured for volatile marker concentration either continuously or periodically. From the exhalation air is extracted at least one measured marker concentration value. Numerous types of breath sampling apparatuses can be used to carry out the method of the present invention.

In one embodiment, the breath sampling apparatus includes a conventional flow channel through which exhalation air flows. The flow channel is provided with a sensor of the subject invention for measuring marker concentration. Furthermore, necessary output elements may be included with the breath sampling apparatus for delivering at least a measured concentration result to the user, if necessary.

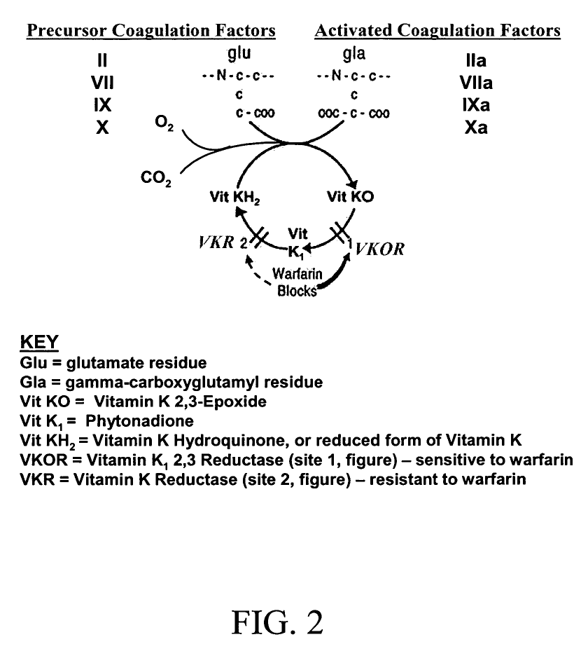

An alarm mechanism may also be provided. An instrument of similar type is shown in FIGS. 1 and 2 of U.S. Pat. No. 5,971,937 incorporated herein by reference.

In another embodiment, once the level of concentration is measured, it is given numerical value (for example, 50 on a scale of 1 to 100). Should the concentration fall below that value, the new value would be indicative of a decrease in concentration. Should the concentration increase beyond that value, the new value would be indicative of an increase in concentration. This numerical scale would allow for easier monitoring of changes in concentration. The numerical scale would also allow for easier translation into control signals for alarms, outputs, charting, and control of external devices (e.g., infusion pump). The upper and lower limits could be set to indicate thresholds such as from ineffective to dangerous therapeutic drug levels.

Compositions

Compositions containing markers and/or nanotechnology in accordance with the present invention can be administered utilizing methods known to the skilled artisan. In one aspect of the invention, the compositions are formulated in admixture with a pharmaceutically acceptable carrier and optionally, with other therapeutic and/or prophylactic ingredients.

In general, it is preferable to administer a pharmaceutical composition of the invention in orally or nasally (i.e., inhalation) administrable form, but formulations may be administered via parenteral, intravenous, intramuscular, transdermal (i.e., topical), buccal, subcutaneous, transmucosal, suppository or other route. Intravenous and intramuscular compositions are preferably administered in sterile saline. One of ordinary skill in the art may modify the compositions of the invention within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification).

According to the present invention, compositions can be delivered to the patient parenterally (i.e., intravenously, intramuscularly). For such forms of administration, the compositions can be formulated into solutions or suspensions, or in lyophilized forms for conversion into solutions or suspensions before use. Sterile water, physiological saline (i.e., phosphate buffered saline (PBS)) can be used conveniently as the pharmaceutically acceptable carriers or diluents. Conventional solvents, surfactants, stabilizers, pH balancing buffers, anti-bacterial agents, chelating agents, and antioxidants can all be used in the these formulations, including but not limited to acetates, citrates or phosphates buffers, sodium chloride, dextrose, fixed oils, glycerine, polyethylene glycol, propylene glycol, benzyl alcohol, methyl parabens, ascorbic acid, sodium bisulfite, and the like. These formulation can be stored in any conventional containers such as vials, ampoules, and syringes.

Sterile injectable solutions of the compositions of the invention can be prepared by incorporating the nanostructure-based assemblies in required amounts in an appropriate solvent with one or a combination of ingredients as required, followed by sterilization. Generally, dispersions are prepared by incorporating the nanostructure-based assemblies into a sterile vehicle that contains a basic dispersion medium, and the other required ingredients. Preparation of sterile powders for sterile injectable solutions include vacuum drying and freeze-drying that yield a powder containing the active ingredient and any desired ingredients to form a sterile solution.

The compositions of the invention can also be delivered orally in enclosed gelatin capsules or compressed tablets. Capsules and tablets can be prepared in any conventional techniques. For example, the active compounds can be incorporated into a formulation, which includes pharmaceutically acceptable carriers such as excipients (i.e., starch, lactose), binders (i.e., gelatin, cellulose, gum tragacanth), disintegrating agents (i.e., alginate, Primogel, and corn starch), lubricants (i.e., magnesium stearate, silicon dioxide), and sweetening or flavoring agents (i.e., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules

Remote Communication System

A further embodiment of the invention includes a communications device in the home (or other remote location) that will be interfaced to sensor technology. The home communications device will be able to transmit immediately or at prescribed intervals directly or over a standard telephone line (or other communication transmittal means such as via satellite communication) the data collected by the data monitor/analyzer device. The communication of the data will allow the user (i.e., physician) to be able to remotely monitor the patient's health. The data transmitted from the home can also be downloaded to a computer where the detected concentrations are stored in a database, and any deviations outside of normal levels would be automatically flagged (i.e., alarm) so that a user (i.e., patient, physician, nurse) could diagnose and/or treat the patient accordingly.

### EXAMPLE 1

### Red Cell (Erythrocyte) Determinations and Measurements

To determine blood hemoglobin concentration by vapor analysis (such as analysis of exhaled breath or headspace using any of the sensor technology described herein such as aptamer biosensors and amplifying fluorescent polymer sensors), the following equation is applied:
Hbᵥ=fₐ(a_{vhb})+f_{b}(b_{vhb})+ . . .
Hbᵥ is the hemoglobin concentration in the circulating blood determined by vapor analysis. The vapor concentrations of volatile markers associated with hemoglobin are represented by a_{vhb}, b_{vhb}, etc. The detection and quantification of a specific volatile marker by a specific sensor is characterized by a unique function, f. A simple example is the linear relationship of Hbᵥ=k·a_{vhb}, where the hemoglobin concentration determined by vapor analysis is directly proportional by the constant k to the concentration of a volatile marker associated with hemoglobin.

Similarly, the concentration of red blood cells (RBC) can be determined by vapor analysis, where the following equation is applied:
RBCᵥ=fₘ(m_{vrbc})+fₙ(n_{vrbc})+ . . .
where RBCᵥ is the red blood cell concentration, a_{vhb}, b_{vhb}, etc. are the vapor concentration of the volatile markers associated with the red blood cell stroma, and f is a unique function which describes the relationship of the vapor concentration of a specific volatile substances associated with red blood cells.

Since the RBC concentration is the main determination of red cell packed cell volume, hematocrit can also be derived using the following equation:
Hctᵥ=h[fₘ(m_{vhct})+fₙ(n_{vhct})+ . . . ]
where h is the function which describes the relationship of RBCᵥ to Hctᵥ.

Using the above determinations of Hbᵥ and Hctᵥ, the mean corpuscular hemoglobin concentration based on vapor analysis (MCHCᵥ) can be derived. MCHCᵥ is useful in categorizing the morphological type of anemia (diagnosed by low Hbᵥ or Hctᵥ).

Determination of plasma volume and blood volume by vapor analysis can be derived using the following equation:
PVᵥ=fₚ(pᵥₚᵥ)+f_{q}(qᵥₚᵥ)+ . . .
where PVᵥ is the plasma volume determined by vapor analysis of the volatile markers Pᵥₚᵥ, qᵥₚᵥ, etc. and fp, fq, etc. are the unique functions describing the relationship of the volatile markers to the plasma volume. The volatile markers related to the plasma volume are those consistently present in whole blood, but not associated with red cell hemoglobin or stroma.

With knowledge of the plasma volume and the hematocrit, Hctᵥ, the following equation can be used to determine blood volume:
BVᵥ=PVᵥ/(1−Hctᵥ)
where BVᵥ is the blood volume determined by vapor analysis.

The subject invention provides systems and methods for performing any one or combination of the following:
1. determine reticulocyte count by sample analysis of markers associated with reticulocytes or other biochemical processes leading to the production of red blood cells;
2. determine erythrocyte survival or the effective duration of red blood cells in the blood by vapor analysis of markers associated with red blood cells or other biochemical processes leading to the clearance of red blood cells from the circulating blood;
3. diagnose malaria (the malaria parasite within the red blood cell) by sample analysis;
4. diagnose histoplasmosis, Bartonella, and other parasitic infections or infestations of red blood cells by sample analysis of associated volatile molecules;
5. diagnose the infection or infestation of red blood cells by clostridia, cholera, tuberculosis, leprosy, and other bacteria by sample analysis;
6. diagnose the infection of red blood cells by viruses (primary atypical pneumonia, infectious mononucleosis, etc);
7. diagnose hemolysis by sample analysis;
8. diagnose hemoglobin types (e.g., A, C, D, E, F, G, H, I, J, M, S) and hemoglobinopathies (e.g., glucose-6-phosphate dehydrogenase defect, pyruvate kinase deficiency, hereditary spherocytosis, hereditary eliptocytosis, thalassemia syndromes, paroxysmal nocturnal hemoglobinuria, etc.) by sample analysis;
9. determine major blood types (e.g. A, B, 0) and detect of other antigens of significance in blood banking on the red blood cell surface (e.g. C, c, D, d, E, e, Kell (K), Duffy (Fy), Kidd (jK), MN, P, Lewis (Le), and Lutheran (Lu) by sample analysis;
10. use intravenously or orally delivered nanotechnology to identify the red blood cell surface antigens that determine blood types. For example, a nanotube with end-caps that release volatile markers on contact with the specific surface blood type antigen. The volatile markers are selected for their volatility, lack of toxicity, and ease of detection in exhaled breath would be released precipitously and detected only if that red blood cell surface antigen were present;
11. spray nanotechnology onto a bleeding wound, wherein the nanotechnology releases either a volatile marker unique to the red blood cell type for vapor detection or a dye marker unique to the blood type present; and
12. determine red blood cell 2,3 diphosoglycerate (2,3 DPG) by sample analysis as an index of oxygen-hemoglobin dissociation.

### EXAMPLE 2

### Platelet (Thrombocyte) Determinations

The systems and methods of the subject invention can also perform any one or combination of the following:
1) determine the platelet concentration in the blood by sample analysis;
2) determine the megakaryocyte concentration in the blood by sample analysis;
3) determine platelet activation by sample analysis;
4) determine platelet maturation by sample analysis;
5) determine platelet production, consumption, and turnover by sample analysis;
6) diagnose intrinsic thrombocytopathies (e.g., Glanzmann's thrombasthenia) or acquired thrombocytopathies by sample analysis;
7) determine the degree of glycoprotein IIb/IIIa receptor blockade and the degree of other platelet glycoprotein receptor occupancy by sample analysis;

### EXAMPLE 3

### White Blood Cell (Leukocyte) Determinations

The subject invention provides systems and methods for performing any one or combination of the following:
1) determine the blood concentration of lymphocytes, polymorphonuclear neutrophils, basophils, eosinophils and monocytes by sample analysis (i.e., the quantity of the particular cell that is circulating per unit volume of blood);
2) determine the ratio of the different “white blood cell” types (lymphocytes, neutrophils, basophils, eosinophils and monocytes), commonly referred to as a differential count, using sample analysis;
3) determine the so-called “left shift” of the polymorphonuclear neutrophil maturation series of Schilling to less mature forms such as early segmented and band neutrophils using sample analysis;
4) determine lymphocyte, polymorphonuclear neutrophil, basophil, eosinophil, and monocyte activity, competence, or capability by sample analysis;
5) diagnose lymphocyte, polymorphonuclear neutrophil, basophil, eosinophil, and monocyte cytopathies by sample analysis;
6) determine lymphocyte, polymorphonuclear neutrophil, basophil, eosinophil, and monocyte maturation by sample analysis;
7) determine lymphocyte, polymorphonuclear neutrophil, basophil, eosinophil, and monocyte production, removal, and turnover by sample analysis;
8) determine the concentration, activity, competence, capability, cytopathy, maturation, production, removal, and turnover of subpopulations of lymphocytes (B-cells, plasma cells, T-cells, helper T-cells, killer T-cells, suppressor T-cells, natural killer cells) and monocytes (phagocytes, macrophages);
9) determine leukocyte surface antigen composition by the administration of nanotechnology designed to contain and release volatile markers when a specific surface antigen is present. For example, a nanotube with end-caps that open on contact with the specific leukocyte surface antigen would contain marker molecules. The marker molecules selected for their volatility, lack of toxicity, and ease of detection in exhaled breath would be released precipitously and detected only if that leukocyte surface antigen were present. Lymphocyte CD4 counts could be measured by exhaled breath marker measurement after ingestion or injection of a composition comprising nanotechnology;
10) diagnose qualitative changes in neutrophils by sample analysis including toxic granulation, the presence of Dohle inclusion bodies, the May-Hegglin anomaly, the Alder-Reilly anomaly, the Pelger-Huet anomaly, and Chediak-Higashi syndrome;
11) diagnose lupus erythematosus by detection and quantitation of LE cells by sample analysis;
12) detect leukemias, lymphomas, and myeloproliferative syndromes by analysis of volatile markers associated with an abnormal cell type or a normal cell type in extraordinarily high concentrations;
13) diagnose malignant histiocytosis by detection and quantitation of hematopoietic stem cells by sample analysis;
14) diagnose reticulum cell sarcoma by detection and quantitation of reticulum cells by sample analysis;
15) diagnose of Hodgkin's Disease by detection and quantification Reed-Sternberg cells by sample analysis;
16) diagnose multiple myeloma by detection and quantitation of proplasmacytes and mature plasma cells by sample analysis;
17) diagnose acute myelogenous leukemia (AML) by detection and quantification myeloblasts by sample analysis;
18) diagnose chronic myelogenous leukemia (CML) by detection and quantification of myelocytes by sample analysis;
19) diagnose acute lymphocytic leukemia (ALL) and lymphoblastic lymphoma by detection and quantification of lymphoblasts by sample analysis;
20) diagnose subacute lymphocytic leukemia and moderately well-differentiated lymphocytic lymphoma by detection and quantification of prolymphocytes by sample analysis;
21) diagnose chronic lymphocytic leukemia (CLL) and well-differentiated lymphocytic lymphoma by detection and quantification of mature lymphocytes by sample analysis;
22) diagnose monocytic leukemia by detection and quantification of monocytes by sample analysis;
23) diagnose other leukemias such as neutrophilic, eosinophilic, basophilic, and thrombocytic leukemias by sample analysis.

### EXAMPLE 4

### Determinations Derived from Cellular Components in Whole Blood

The systems and methods of the subject invention can also accomplish diagnosis of non-hematological malignancy by sample analysis of volatile markers associated with circulating malignant cells. Solid, non-hematological tumors that exhibit remote metastases by hematological spread are included in those malignancies that can be detected using the subject invention.

In certain embodiments, the sensor technology can be designed to be an integral part of the sample container. For example, a combined sensor/container of the subject invention can be manufactured so that patients provide a sample of blood into the sensor/container and detection of volatile markers in the headspace can be signaled to the user immediately after the container is capped. This, however, is not a limitation on the invention as samples of bodily fluids can be sampled immediately or stored for future application of headspace to a sensor according to the subject invention.

### EXAMPLE 5

### Assessment of Protein Function Using Breath-based Sensor Systems of the Invention

To illustrate how the breath-based sensor technologies outlined in this patent application can be used to determine protein function (e.g., enzyme activity), a specific example is highlighted hereafter, which is of enormous clinical and economic importance to the global health care sector. In particular, a non-invasive, real time point-of-care (POC) breath-based method of monitoring the degree of enzyme (vitamin K epoxide reductase, VKOR) inhibition caused by warfarin (see FIG. 2), which leads to anticoagulation by depleting active levels of specific vitamin K-dependent coagulation proteins, will be described.

The method described herein of assessing warfarin-induced inhibition of VKOR activity will 1) complement or even replace current lab measures of warfarin anticoagulation such as the International Normalized Ratio (INR) or the prothrombin test (PT), 2) potentially lead to much safer and more efficacious clinical utilization of warfarin, 3) identify those subjects with quantitative and/or qualitative defects in VKOR function (e.g., genetic abnormalities of VKOR, drug-acquired dysregulation of VKOR) that could lead to complications of warfarin use (i.e., too much anticoagulation leading to hemorrhage or too little anticoagulation leading to thrombotic sequalae), and 4) allow patients to more readily monitor their degree of anticoagulation at home and reduce the need for frequent costly visits to “INR clinics.”

The subject invention overcomes these deficiencies in the use of warfarin and other related anticoagulants. According to the invention, a composition comprising nanoparticles is administered to a patient who is given warfarin (or similar anticoagulant). The nanoparticles comprise a detector for VKOR or VKOR substrates and/or metabolites, and volatile markers. Upon detection of VKOR or VKOR metabolites/substrates, the volatile markers are released from the nanoparticles as described herein. By continuously monitoring the end-tidal exhaled breath for volatile marker concentration, the clinician can monitor anticoagulation levels in the patient. This will allow the healthcare provider to maintain the patient in an appropriate anticoagulation level and overcome many of the complications related to using warfarin for long periods. The accurate real time assessment of VKOR activity would be a significant addition to the clinical tools used to properly manage warfarin anticoagulation.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

We claim:

1. A method for detecting volatile markers associated with hematological conditions or diseases, said method comprising the steps of:
(a) collecting a sample of blood from a patient into a container, wherein said container includes a cover and provides room for a headspace sample;
(b) analyzing the headspace sample with sensor technology to determine the presence of the volatile markers associated with hematological conditions or diseases.

2. The method according to claim 1, wherein the sensor technology is based on the group consisting of polynucleotides, peptides, synthetic receptors, polymeric unnatural biopolymers, imprinted polymers, small fragments of DNA or RNA.

3. The method according to claim 2, wherein the sensor technology is based on aptamers, enzymes, antibodies, polythioureas, or polyguanidiniums.

4. The method according to claim 1, wherein the sensor technology is selected from the group consisting of surface-acoustic-wave sensors; fluid sensor technology; semiconductive gas sensors, mass spectrometers; IR, V, visible and fluorescence spectrophotometers; conductive-polymer gas-sensors; aptamer biosensors; and amplifying fluorescent polymer sensors.

5. The method according to claim 1, wherein the sensor technology comprises:
(a) a surface-acoustic wave (SAW) sensor capable of detecting the presence of a marker in a sample of bodily fluid, wherein the SAW sensor responds to the marker by a shift in the resonant frequency;
(b) an oscillator circuit having the SAW sensor as an active feedback element;
(c) a frequency counter in communication with said oscillator circuit to measure oscillation frequency which corresponds to resonant frequency of the SAW sensor; and
(d) a processor for comparing the oscillation frequency with a previously measured oscillation frequency of the marker and determining presence and concentration of the marker therefrom.

6. The method according to claim 1, wherein the sensor technology comprises:
(a) a sensor having an array of polymers capable of detecting the presence of the marker in the sample of bodily fluid, wherein said sensor responds to the marker by changing the resistance in each polymer resulting in a pattern change in the sensor array;
(b) a processor for receiving the change in resistance, comparing the change in resistance with a previously measured change in resistance, and identifying the presence of the marker from the pattern change and the concentration of the marker from the amplitude.

7. The method according to claim 1, wherein the volatile markers are associated with hematological conditions or diseases selected from the group consisting of: Afibrinogenemia, Agammaglobulinemia, Anemia (i.e., aplastic anemia, hemolytic anemia, congenital nonspherocytic anemia, megaloblastic anemia, pernicious anemia, sickle cell anemia, Fanconi Anemia), Angiolymphoid Hyperplasia with Eosinophilia, Antithrombin III Deficiency, Bernard-Soulier Syndrome, Blue Rubber Bleb Nevus Syndrome, Chediak-Higashi Syndrome, Cryoglobulinemia, Disseminated Intravascular Coagulation, Eosinophilia, Erdheim-Chester Disease, Erythroblastosis, Fetal—Evans Syndrome, Factor V Deficiency, Factor VII Deficiency, Factor X Deficiency, Factor XI Deficiency, Factor XII Deficiency, Giant Lymph Node Hyperplasia, Hemoglobinuria, Paroxysmal—Hemoglobinuria, Paroxysmal—Hemophilia A, Hemophilia B, Hemorrhagic Disease of Newborn, Histiocytosis, Langerhans-Cell, Non-Langerhans-Cell, Job's Syndrome, Leukopenia, Lymphadenitis, Lymphangiomyomatosis, Lymphedema, Methemoglobinemia, Myelodysplastic Syndromes, Myelofibrosis, Myeloid Metaplasia, Myeloproliferative Disorders, Neutropenia, Paraproteinemias, Platelet Storage Pool Deficiency, Polycythemia Vera, Protein C Deficiency, Protein S Deficiency, Purpura (i.e., thrombocytopenic purpura, thrombotic thrombocytopenic purpura), Sarcoidosis, Spherocytosis, Splenic Rupture, Thalassemia, Thrombasthenia, Thrombocytopenia, Waldenstrom Macroglobulinemia, and von Willebrand Disease.

8. The method according to claim 1, further comprising the step of treating the headspace to concentrate the volatile markers in the headspace.

9. The method according to claim 8, wherein the headspace is treated to remove water vapor.

10. The method according to claim 1, further comprising the step of treating the sample of blood to concentrate the volatile markers in the headspace.

11. The method according to claim 10, wherein the sample of blood is heated to a temperature between 50° F. to 110° F. to promote increased concentration of volatile markers in the headspace.

12. The method according to claim 1, further comprising the step of allowing the sample of blood to equilibrate in the container.

13. The method according to claim 1, wherein the sample of blood includes materials selected from the group consisting of: erythrocytes, hemoglobin, leukocytes, thrombocytes, plasma, sugars, lipids, vitamins, minerals, proteins, hormones, enzymes, and antibodies.

14. The method of claim 1, further comprising the step of administering a composition to a patient comprising nanoparticles, wherein each nanoparticle comprises a detector and a volatile marker, wherein detection of a target biomarker associated with a hematological condition or disease causes the release of the volatile marker for detection by the sensor technology.

15. The method of claim 14, wherein the detector is selected from the group consisting of an aptamer; an antibody, and a protein.

16. The method of claim 14, wherein the volatile marker is selected from the group consisting of benzodiazepines; Phenobarbitals; dimethyl sulfoxide (DMSO), acetaldehyde, acetophenone, anise, benzaldehyde, benzyl alcohol, benzyl cinnamate, cadinene, camphene, camphor, cinnamon, garlic, citronellal, cresol, cyclohexane, eucalyptol, and eugenol, eugenyl methyl ether.

17. The method of claim 14, wherein the volatile marker is selected from GRAS compounds consisting of sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

18. A method for non-invasive, real-time assessment of anticoagulation levels in a patient, said method comprising the steps of:
a) administering to a patient an anticoagulant;
b) administering a composition comprising nanoparticles, wherein each nanoparticle comprises a detector and a volatile marker, wherein detection of a target biomarker associated with anticoagulation levels causes the release of the volatile marker for detection by the sensor technology;
c) collecting a bodily fluid sample from the patient;
d) applying sensor technology to the bodily fluid sample to determine the presence of volatile markers associated with anticoagulation levels; and
e) determining anticoagulation levels in the patient based on the concentration of volatile markers in the bodily fluid sample.

19. The method of claim 18, wherein the anticoagulant is warfarin.

20. The method of claim 19, wherein the detector has an affinity for VKOR or a metabolite or substrate of VKOR.

21. The method of claim 18, wherein the bodily fluid sample is exhaled breath or blood.

22. The method of claim 21, wherein the sample of blood is collected in a container, wherein said container includes a cover and provides room for a headspace sample; and wherein the sensor technology is applied to the headspace.

23. The method according to claim 18, wherein the sensor technology is selected from the group consisting of surface-acoustic-wave sensors; fluid sensor technology; semiconductive gas sensors, mass spectrometers; IR, UV, visible and fluorescence spectrophotometers; conductive-polymer gas-sensors; aptamer biosensors; and amplifying fluorescent polymer sensors.
